(19) United States
(12) Patent Application Publication
(10) Pub. No.: US 2013/0331765 A1
(43) Pub. Date: Dec. 12, 2013

(54) TREATING ERYTHEMATOTELANGIECTATIC ROSACEA OR PAPULOPUSTULAR ROSACEA WITH NARROW-BAND INFRARED LIGHT RADIATION AND RADIATION KITS THEREFOR

(71) Applicant: Seung Yoon Lee, Seongnam-si (KR)

(72) Inventor: Seung Yoon Lee, Seongnam-si (KR)

(21) Appl. No.: 13/889,244

(22) Filed: May 7, 2013

Related U.S. Application Data
(63) Continuation of application No. 12/386,113, filed on Apr. 14, 2009, now abandoned.
(60) Provisional application No. 61/124,056, filed on Apr. 14, 2008.

(51) Int. Cl. A61N 5/06

(52) U.S. Cl. CPC A61N 5/0616
USPC 604/20; 607/88

(57) ABSTRACT

A method of treating erythematotelangiectatic rosacea or papulopustular rosacea in a subject comprises exposing the subject's skin in need thereof to narrow-band infrared radiation in an effective dose to treat erythematotelangiectatic rosacea or papulopustular rosacea and essentially not to cause photothermolysis of the skin.

### RELATED APPLICATION

This application claims the benefit of U.S. Provisional Application No. 61/124,056, filed on Apr. 14, 2008. The entire teachings of the above application are incorporated herein by reference.

### BACKGROUND OF THE INVENTION

Rosacea is a chronic disease that affects the skin and sometimes the eyes. Symptoms include skin redness, pink bumps (papules), bumps containing pus (pustules), pimples, abnormal proliferation and dilation of superficial blood vessels (telangiectasia), and, in the advanced stages, thickened skin. The National Institute of Arthritis and Musculoskeletal and Skin Diseases (NIAMS) reports that approximately 14 million people in the US suffer from rosacea. Several subtypes of rosacea are known in the art, including erythematotelangiectatic rosacea, papulopustular rosacea, phymatous rosacea and ocular rosacea.

Typically, in erythematotelangiectatic type rosacea, central facial flushing, often accompanied by burning or stinging, is the predominant sign. The redness usually spares the periocular skin. The erythematous areas of the face at times appear rough presumably due to chronic, low-grade dermatitis with inflammation. Frequent triggers to flushing include acutely felt emotional stress, hot drinks, alcohol, spicy foods, exercise, cold or hot weather, and hot baths and showers. These patients also report that the burning or stinging is exacerbated when topical agents are applied.

Typically, papulopustular rosacea is the classic presentation of rosacea. Patients are generally women of middle age who predominately present with a red central portion of their face that contains small erythematous papules surmounted by pustules. One may elicit a history of flushing. Telangiectasias are likely present but may be difficult to distinguish from the erythematous background in which they exist.

The etiology of rosacea is not elucidated yet. Some possible causes that have been suggested to be related to development of rosacea are inherited abnormalities in cutaneous vascular homeostasis, exposure to sunlight, dermal matrix degeneration, chemical and ingested agents, abnormalities of sebaceous gland, certain microbial organisms such as Demodex and Helicobacter pylori. In addition, it has been proposed that those who blush frequently may be more likely to develop rosacea, and research has shown that rosacea is a disorder where blood vessels dilate too easily, resulting in flushing and redness. While the cause is unknown and there is no cure, the signs and symptoms of the disorder can be managed.

So far, erythematotelangiectatic and papulopustular rosacea has been treated with various treatment modalities including laser treatments for telangiectatic lesions, low dose systemic antibiotics such as doxycycline, and topical agents such as topical metronidazole or azelaic acid, with variable success rate. Especially, pulsed dye laser or potassium titanyl phosphate (KTP) laser (which is typically based on selective photothermolysis) has been widely used for telangiectatic lesions regardless whether it is resulted from rosacea or other conditions. In selective photothermolysis, typically, chromophores (e.g. hemoglobin in blood vessels) absorb high-power energy of pulses of light from laser source. Then the light energy converts to heat energy and the resultant thermal injury causes destruction of the target chromophore. When the chromophore is hemoglobin of blood vessels, the vessels are also destroyed by the thermal damage or by resultant inflammatory process. Intense pulsed light (IPL) can also be used in a similar context as the pulsed dye laser or KTP laser in the treatment of rosacea.

Although effective for many telangiectatic lesions of rosacea, laser or IPL treatment for rosacea can cause some side effects such as purpura, hyperpigmentation, hypopigmentation, burn and scarring. These side effects are especially of concern to dark-skinned individuals and people who have tanned skin, as melanin is also one of chromophores that absorb light at the wavelength(s) employed such lasers and IPL. Because these devices produce high-power, pulsed light energy that can create photothermolysis of, hence, thermal injury to skin components including melanin-containing epidermis and adjacent structures, they can cause these adverse effects mentioned above, and therefore, are not suitable for some patients with dark skin or tanned skin. Additionally, some rosacea lesions that have erythematous background and fine vessels but not prominent dilated vessels sometimes tend to be resistant to laser treatment with pulsed dye lasers or KTP lasers, possibly due to lack of sufficient chromophores in blood vessels (e.g., hemoglobin) in such lesions and the presence of melanin particles in adjacent skin that compete for absorption of laser energy. These limitations of current laser and IPL treatment make it difficult to treat erythematotelangiectatic rosacea.

The treatment of papulopustular rosacea usually involves uses of topical agent such as topical metronidazole, azelaic acid, and low dose oral antibiotics such as doxycycline. The mechanism of their efficacy is not fully understood, but anti-inflammatory effect of such agents is believed to play a role. However, their use may cause some side effects such as skin irritation and bacterial resistance to the antibiotics used. The outcome of these treatments is variable according to the treatment protocols and the patients' condition.

Thus, there is a need for developing a method for treating rosacea, such as erythematotelangiectatic type rosacea and papulopustular rosacea, in particular in a non-invasive manner, e.g., without photothermolysis of the skin under treatment or skin irritation.

### SUMMARY OF THE INVENTION

The present invention generally relates to a method of treating erythematotelangiectatic rosacea or papulopustular rosacea with narrow-band infrared radiation, and to a kit therefor.

In one embodiment, the present invention is directed to a method of treating erythematotelangiectatic rosacea or papulopustular rosacea in a subject. The method comprises exposing the subject's skin in need thereof to narrow-band infrared radiation at a wavelength(s) in a range of between 790 nm and 900 nm and having a band width of between 0 nm and 20 nm, in an effective dose to treat erythematotelangiectatic rosacea or papulopustular rosacea and essentially not to cause photothermolysis of the skin.

In another embodiment, the present invention is directed to a method of treating erythematotelangiectatic rosacea or papulopustular rosacea in a subject. The method comprises exposing the subject's skin in need thereof to narrow-band infrared radiation at a wavelength(s) in a range of between 790 nm and 900 nm and having a band width of between 0.1 nm and 20 nm, in an effective dose to treat erythematotelangiectatic rosacea or papulopustular rosacea.

In yet another embodiment, the present invention is directed to a kit comprising a radiation source generating narrow-band infrared radiation at a wavelength(s) in a range of between 790 nm and 900 nm, the narrow-band infrared radiation having a band width of between 0 nm and 20 nm and having a power density of between 1 mW/cm² and 100 mW/cm², and a manual instructing a user how to use the narrow-band infrared radiation for treating erythematotelangiectatic rosacea or papulopustular rosacea.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts a kit of the invention that includes a radiation device generating narrow-band infrared radiation employed in the invention, and a manual instructing a user how to use the narrow-band infrared radiation for treating a subject with erythematotelangiectatic rosacea or papulopustular rosacea.

FIG. 2A is a schematic drawing of one embodiment of a radiation device and a protection shield that can be employed in the invention.

FIG. 2B is a schematic drawing of protective goggles that can be employed in the invention for protecting the retinae of the eyes of a subject to be treated with the narrow-band infrared radiation of the invention from direct illumination at the wavelength(s) of the narrow-band infrared radiation.

FIG. 2C is a schematic drawing of another embodiment of a radiation device, a protection shield and a pair of protective goggles, which can be employed in the invention.

FIG. 2D is a schematic drawing of a patient under one embodiment of the narrow-band infrared radiation treatment of the invention.

FIG. 3 is a photograph showing a cheek of a patient, who has telangiectasia on the cheek, prior to the narrow-band infrared radiation treatment of the invention.

FIGS. 4 and 5 are follow-up photos of the cheek of the patient of FIG. 3 three months and seventeen months after the narrow-band infrared radiation treatment of the invention, respectively, which show substantial reduction of telangiectasia on the patient's cheek after the narrow-band infrared radiation treatment.

### DETAILED DESCRIPTION OF THE INVENTION

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

The invention employs narrow-band radiation at a wavelength(s) in a range of between 790 nm and 900 nm and having a band width of between 0 nm and 20 nm, or alternatively between 0.1 nm and 20 nm, in an effective dose to treat erythematotelangiectatic rosacea or papulopustular rosacea in a subject.

Rosacea is generally defined by persistent erythema of the central portion of the face lasting for at least 3 months. Supporting criteria include flushing, papules, pustules, and telangiectasias on the convex surfaces. Secondary characteristics are burning and stinging, edema, plaques, a dry appearance, ocular manifestations, and phymatous changes. The prevalence of these findings designates the subclassification of the presentation and, additionally, the therapeutic options. The diagnosis of rosacea is a clinical diagnosis. Before making the diagnosis of rosacea, skin biopsy may be necessary to exclude other disease states that mimic the clinical presentation of rosacea, for example to exclude polycythemia vera, connective tissue diseases (eg, lupus erythematosus, dermatomyositis, mixed connective tissue disease), carcinoid, mastocytosis, long-term application of topical steroids, contact dermatitis and photosensitivity.

Typically, in erythematotelangiectatic type rosacea, central facial flushing, often accompanied by burning or stinging, is the predominant sign. The redness usually spares the periocular skin. The erythematous areas of the face at times appear rough presumably due to chronic, low-grade dermatitis with inflammation. Frequent triggers to flushing include acutely felt emotional stress, hot drinks, alcohol, spicy foods, exercise, cold or hot weather, and hot baths and showers. These patients also report that the burning or stinging is exacerbated when topical agents are applied.

Typically, papulopustular rosacea is the classic presentation of rosacea. Patients are generally women of middle age who predominately present with a red central portion of their face that contains small erythematous papules surmounted by pustules. One may elicit a history of flushing. Telangiectasias are likely present but may be difficult to distinguish from the erythematous background in which they exist.

“Narrow-band” radiation, as used herein, means radiation at a wavelength or wavelengths having a band width between 0 nm and 20 nm, such as between 0.1 nm and 20 nm. It is noted that the term “between 0 nm and 20 nm” includes “0 nm” and “20 nm.” For example, narrow-band radiation at a wavelength(s) having a band width of 20 nm means that the radiation at the wavelength(s) has, for example, a deviation of ±10 nm. Similarly, narrow-band radiation at a wavelength(s) having a band width of 0 nm means that the radiation at the wavelength(s) has a deviation of ±0 nm.

As used herein a “subject” is a mammal, preferably a human. Subject and patient are used interchangeably.

“Treatment” or “treating” refers to both therapeutic and prophylactic treatment, and also includes any improvement of the condition being treated with the narrow-band infrared radiation treatment compared with the absence of such treatment.

As used herein, the “effective dose” is a quantity that results in a beneficial clinical outcome of, or exerts an influence on, the condition being treated with the narrow-band infrared radiation treatment compared with the absence of such treatment. For example, the “effective dose” can be a dose sufficient to treat erythematotelangiectatic rosacea or papulopustular rosacea in a subject after each dose or after a plurality of consecutive such doses. When a plurality of consecutive doses are employed, the doses are typically repeated at intervals of from 0.5 day (twice per day) to 10 days. Specifically, the doses are repeated at intervals of from one day to 4 days, such as 2 days or 3 days. Alternatively, each irradiant dose is up to 7 days apart, such as 1 day apart, 2 days apart, 3 days apart or 4 days apart. Alternatively, each irradiant dose is 1 day apart, 2 days apart or 3 days apart. The intervals can be the same length or different lengths. In one specific embodiment, the intervals are the same length.

Generally, an effective dose of the narrow-band infrared radiation depends, in each case, upon several factors, e.g., the skin types, age, gender and condition of the subject to be treated, among others.

Generally, the effective dose essentially does not cause photothermolysis of the skin. Selective photothermolysis is a photothermolytic reaction by which a target chromophore is selectively damaged or destroyed by light, resulting in destruction of the target chromophore or necrosis of the cells that contain the target chromophore. Photothermolysis generally occurs when the following three fundamental conditions are met:

Wavelength: specific wavelength that can be absorbed by the target molecule;

Pulse duration: pulse duration of the pulsed light from a laser that is shorter than the thermal relaxation time (TRT) of the target (TRT: TRT is the time taken for the target to dissipate about 63% of the incident thermal energy); and

Fluence (energy density, J/cm²): a sufficient fluence (energy density; the amount of energy per unit area) to create the thermal damage enough to destroy the target.

In one example, meeting the above-mentioned three conditions, when the pulsed light from a laser is absorbed by a given target within time duration shorter than the TRT of the target (thus, the pulse duration of the pulsed light should be shorter than the TRT of the target), the target cannot dissipate the heat energy to the adjacent structures before the sufficient amount of energy to destroy it accumulates in it, and therefore, is destroyed by the thermal damage.

Typically, photothermolysis can occur with a light source that can produce short pulses. Generally, the TRT (thermal relaxation time) is so short that only pulsed light sources, such as lasers or intense pulsed light (IPL), can produce light that has pulse duration sufficiently shorter than TRT of the given target (e.g., TRT of blood vessels).

In general, a fluence that causes photothermolysis varies depending upon the types of the target, TRT of the target, depth of the target, type of lasers, skin phototypes of the subjects, and many other things, because, at least in part, power sufficient for photothermolysis varies depending upon the target, laser type, depth of the target, skin phototypes, etc. Such power that can cause photothermolysis generally cannot be produced with a light-emitting diode (LED) light source or with a light source which cannot produce pulses of light. Thus, with an LED light source or a low level laser or a low level light therapy device, which cannot produce high-power pulses of light, selective photothermolysis of any given components of the skin, including blood vessels, generally does not occur.

In one embodiment, the narrow-band infrared radiation employed in the invention does not meet at least one of the above-mentioned three requirements for photothermolysis.

In another embodiment, the narrow-band infrared radiation employed in the invention is generated from an LED light source or a low level laser. Alternatively, the narrow-band infrared radiation employed in the invention is generated from a low level light therapy device which does not generate pulses of light that has power high enough to cause photothermolysis of skin components.

Typically, in the disclosed methods, the skin of the subject is exposed to a plurality of exposures. The exposures can be repeated for any time period, as long as the subject does not experience any side effect, such as photosensitity. The plurality of exposures are collectively referred to as a “treatment period.” The treatment period can be between one week and 12 weeks, or between two weeks and 8 weeks, such as two, three, four, five or six weeks. Alternatively, the treatment period can be longer than 12 weeks.

In one embodiment, the skin is exposed to the narrow-band infrared radiation one, two, three, four, five, six or seven times per week during the treatment period. In another embodiment, the skin is exposed to the narrow-band infrared radiation four, five, six or seven times per week during the treatment period.

In another embodiment, a layer of a gel, cream or lotion is applied on the skin prior to the skin exposure to the narrow-band infrared radiation. Thus, in this embodiment, the skin is exposed to the narrow-band infrared radiation through the gel, cream or lotion layer. In one specific embodiment, the gel, cream or lotion has at least 70% transparency at the narrow-band infrared radiation. In another specific embodiment, the gel, cream or lotion has at least 90% transparency at the narrow-band infrared radiation. In yet another specific embodiment, a layer of a transparent gel having, for example, at least 70% transparency, particularly at least 90% transparency, at the narrow-band infrared radiation is applied to the skin prior to the skin exposure to the narrow-band infrared radiation. In yet another specific embodiment, the transparent gel is water-based. In yet another specific embodiment, the water-based transparent gel comprises hyaluronic acid. In yet another specific embodiment, the water-based transparent gel consists essentially of water and hyaluronic acid.

In the invention, the narrow-band infrared radiation is at a wavelength(s) in a range of between 790 nm and 900 nm. Alternatively, the narrow-band infrared radiation is at a wavelength(s) in a range of between 800 nm and 860 nm. Alternatively, the narrow-band infrared radiation is between 820 nm and 840 nm. Alternatively, the narrow-band infrared radiation is at a wavelength(s) in a range of between 825 nm and 835 nm. Alternatively, the narrow-band infrared radiation is at 830 nm.

Typically, the narrow-band infrared radiation has a band width of between 0 nm and 20 nm, or between 0.1 nm and 20 nm. Specifically, in any one of the embodiments described in the previous paragraph, the band width of the narrow-band infrared radiation is between 0 nm and 15 nm, such as 0 nm, 6 nm, 10 nm, 12 nm or 15 nm. More specifically, in any one of the embodiments described in the previous paragraph, the band width of the narrow-band infrared radiation is between 0 nm and 12 nm. Alternatively, in any one of the embodiments described in the previous paragraph, the band width of the narrow-band infrared radiation is between 0.1 nm and 12 nm, such as 10 nm. Alternatively, in any one of the embodiments described in the previous paragraph, the band width of the narrow-band infrared radiation is between 0.1 nm and 1 nm.

The narrow-band infrared radiation employed in the invention generally has power density in a range of between 1 mW/cm² and 100 mW/cm². In one specific embodiment, the power density is in a range of between 1 mW/cm² and 75 mW/cm². In another specific embodiment, the power density is in a range of between 1 mW/cm² and 50 mW/cm². In yet another specific embodiment, the power density is in a range of between 1 mW/cm² and 30 mW/cm². In yet another specific embodiment, the power density is in a range of between 1 mW/cm² and 15 mW/cm².

In any one of the embodiments described above, including the embodiments described in the three previous paragraphs, specifically, the narrow-band infrared radiation employed in the invention has energy density in a range of between 3 J/cm² and 180 J/cm². Alternatively, the energy density is in a range of between 3 J/cm² and 150 J/cm². Alternatively, the energy density is in a range of between 3 J/cm² and 120 J/cm². Alternatively, the energy density is in a range of between 3 J/cm² and 100 J/cm². Alternatively, the energy density is in a range of between 3 J/cm² and 70 J/cm². Alternatively, the energy density is in a range of between 3 J/cm² and 50 J/cm². Alternatively, the energy density is in a range of between 3 J/cm² and 30 J/cm².

The skin exposure to the narrow-band infrared radiation per each dose can last for any suitable time period as long as it can cause treatment of erythematotelangiectatic rosacea or papulopustular rosacea of the patient's skin after each dose or after a plurality of such doses, and essentially not to cause photothermolysis of skin components. In one example, the skin exposure to the narrow-band infrared radiation per each dose lasts for less than 20 minutes, such as between 5 minutes and 20 minutes, or between 5 minutes and 15 minutes. Alternatively, the skin exposure to the narrow-band infrared radiation per each dose can last for more than 20 minutes, for example, between 20 minutes and 60 minutes or between 20 minutes and 40 minutes.

For the narrow-band infrared radiation employed in the invention, any suitable radiation source can be employed, including relatively low-power laser and low level light therapy devices and LEDs known in the art. Specifically, the narrow-band infrared radiation employed in the invention is non-coherent radiation. More specifically, the narrow-band infrared radiation employed in the invention is generated by an LED device.

Generally, prior to performing the narrow-band infrared radiation treatment of the invention, it is generally checked whether or not the subject to be treated has any conditions where exposure to light may affect the health of her/his skin, such as photosensitive condition, especially in regards to any possibility of photosensitivity. Examples of such conditions include: recent history (e.g., within one weak) of systemic or topical photodynamic therapy involving any photosensitizer that has the absorption peaks within or near the range of the near infrared light and the use of such photosensitizer for any other purposes; any photosensitive condition, such as disease (e.g. systemic lupus erythematosus, certain types of porphyria (erythropoietic porphyria, erythropoietic protoporphyria, porphyria cutanea tarda, variegate porphyria, hereditary coproporphyria, hepatoerythropoietic porphyria), polymorphous light eruption, hydroa vacciniforme, and other conditions that can cause photosensitivity), drugs (e.g. tetracycline, fluoroquinolones, ibuprofen, amiodarone, phenothiazine, furosemide, hydrochlorothiazide, retinoic acid, isotretinoin, etc.). For example, it is generally checked whether or not the subject to be treated has photosensitive condition, especially in regards to any possibility of photosensitivity, such as disease (e.g. systemic lupus erythematosus, certain types of porphyria (erythropoietic porphyria, erythropoietic protoporphyria, porphyria cutanea tarda, variegate porphyria, hereditary coproporphyria, hepatoerythropoietic porphyria), polymorphous light eruption, hydroa vacciniforme, and other conditions that can cause photosensitivity), drugs (e.g. tetracycline, fluoroquinolones, ibuprofen, amiodarone, phenothiazine, furosemide, hydrochlorothiazide, retinoic acid, isotretinoin, etc.), recent history of photodynamic therapy using 5-aminolevulinic acid or methyl-5-aminolevulinic acid or photofrin or other photosensitizers. If the subject has one or more of these conditions or other photosensitive conditions, it is recommended for the subject to consult her/his doctor regarding whether or not, and/or when, she/he can take the narrow-band infrared radiation treatment of the invention. Also, even if the subject does not have any of the above-mentioned conditions, but if the subject has any photosensitivity condition to the visible light (e.g, between 400 nm and 670 nm), it is generally recommended for the subject to consult her/his doctor regarding whether or not, and/or when, she/he can take the narrow-band infrared radiation treatment of the invention.

The invention also includes a kit comprising a radiation device that includes a radiation source generating the narrow-band infrared radiation employed in the narrow-band infrared radiation methods described above. Specifically, the narrow-band infrared radiation has power density in a range of between 1 mW/cm² and 100 mW/cm². Alternatively, the power density is between 1 mW/cm² and 75 mW/cm². Alternatively, the power density is between 1 mW/cm² and 50 mW/cm². Alternatively, the power density is between 1 mW/cm² and 30 mW/cm². Alternatively, the power density is between 1 mW/cm² and 15 mW/cm². The kit further comprises a manual instructing a user how to use the narrow-band infrared radiation for the narrow-band infrared irradiation treatment to treat erythematotelangiectatic rosacea or papulopustular rosacea on the skin of a subject. Features, including specific features, of the narrow-band infrared irradiation treatment using the kit are as described above for the methods of the invention.

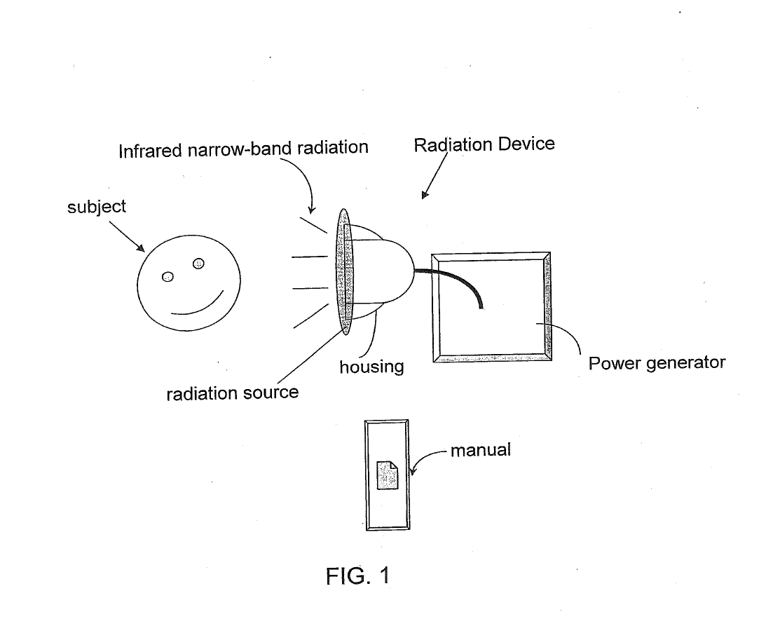

FIG. 1 shows one embodiment of a kit of the invention, comprising a radiation device, such as an LED device, and a manual instructing a user how to use the narrow-band infrared radiation for the narrow-band infrared irradiation treatment to treat erythematotelangiectatic rosacea or papulopustular rosacea on the skin of a subject. The housing of the radiation device of the figure can include any suitable radiation source, such as one or more LEDs.

In one embodiment of a kit of the invention, the radiation device is an LED light device or a low level laser. In another embodiment of a kit of the invention, the radiation device is a low level light therapy device which does not generates pulses of light whose power is high enough to cause photothermolysis of skin components.

In a specific embodiment, the radiation source is a non-coherent radiation source, such as an LED device that includes one or more LEDs.

In another embodiment, the manual included in a kit of the invention further comprises instructions about distance between the skin of the subject and the radiation source during the narrow-band infrared radiation treatment, duration time per single treatment of the narrow-band infrared radiation and frequency of the narrow-band infrared radiation treatment, and warning about conditions where exposure to the narrow-band infrared irradiation may affect the health of the subject's skin. Specific examples of such conditions are as described above. In a further specific embodiment, the warning also recommends users or subjects that they should seek professional advice as to whether the subject(s) to be treated have any photosensitive condition prior to using the kit for the narrow-band infrared radiation treatment, if they do not have prior knowledge about this.

In a specific embodiment, the kit further comprises a pair of goggles that are specifically designed to protect the retinae of the eyes of the subject to be treated with the narrow-band infrared radiation from direct illumination at the wavelength(s) of the narrow-band infrared radiation. Specifically, the goggles have color and/or optical density to essentially block light at the wavelength(s) of the narrow-band infrared radiation.

In another specific embodiment, the manual of the kit includes warning that direct exposure of the eyes to the narrow-band infrared may harm the eyes. In yet another specific embodiment, the manual further provides guidance that any people who do not wear suitable protective goggles must not be exposed to the narrow-band infrared radiation. One example of such protecting guidance is to recommend a user to use the narrow-band infrared irradiation treatment alone in a room which is closed (locked or not), and/or to put a warning sign on the door of the room that a suitable protective goggles should be worn before entering the room.

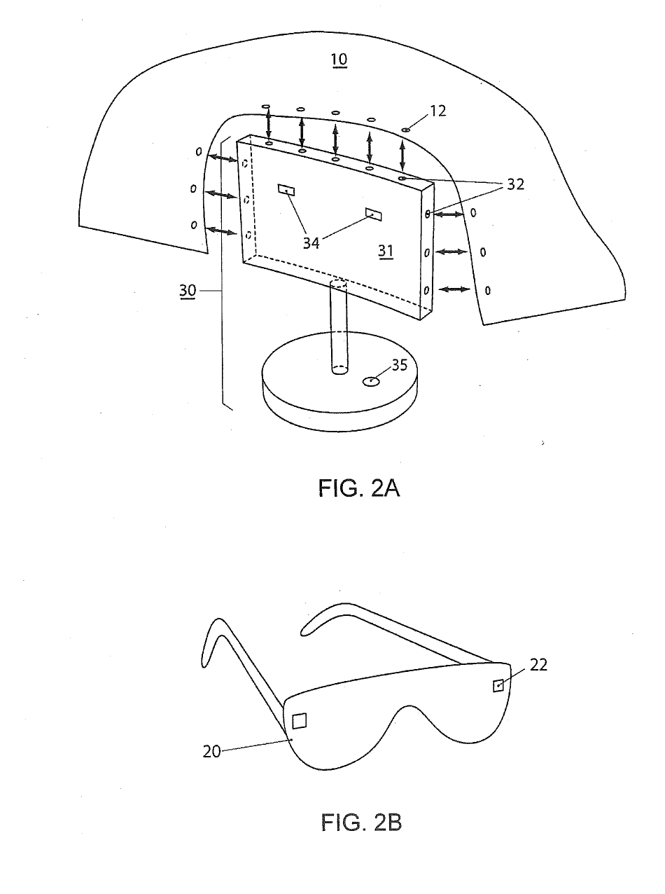

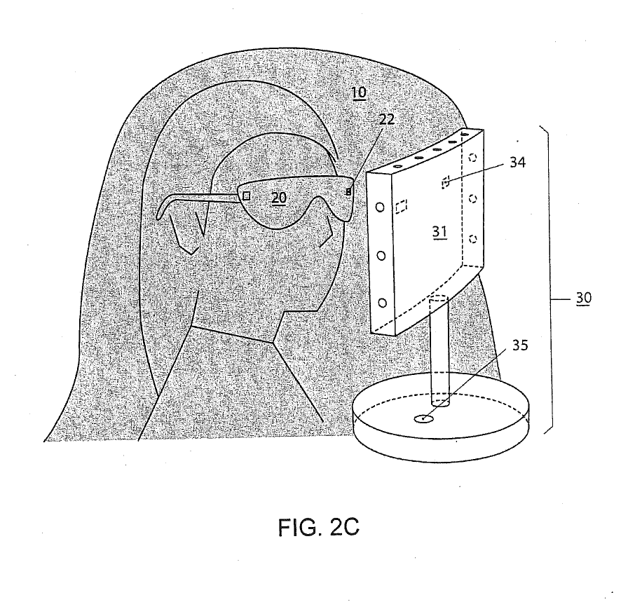

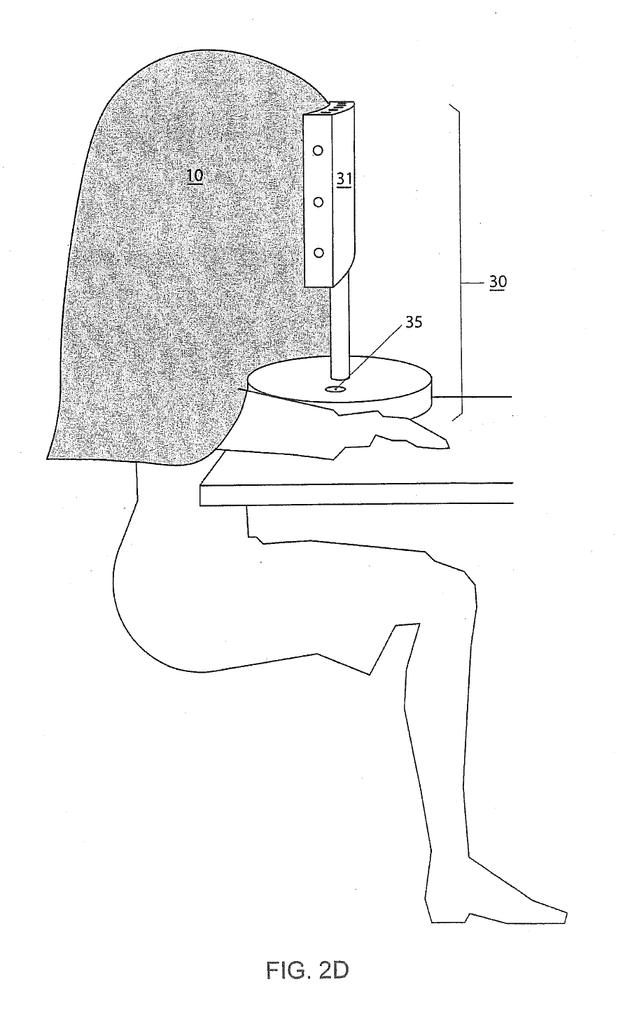

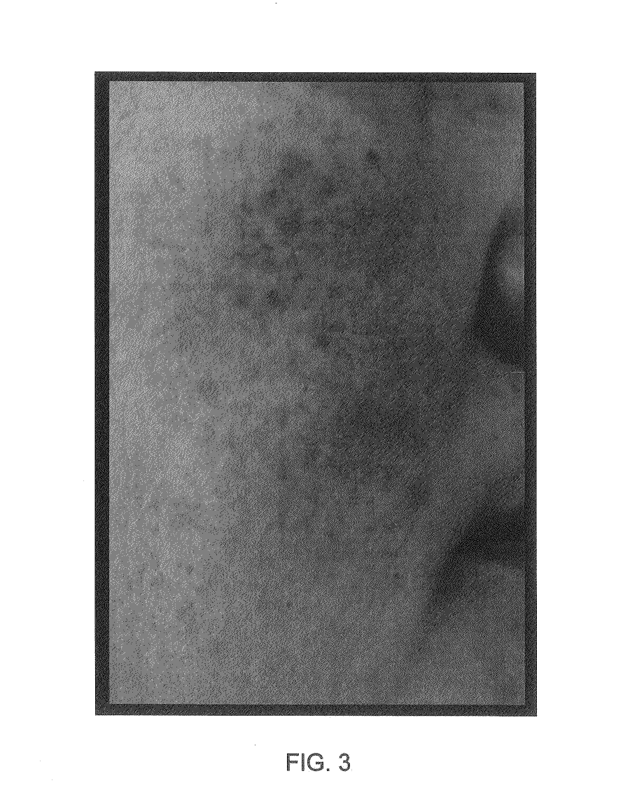

In yet another specific embodiment, a kit of this invention comprises a protective shield (e.g., protective shield 10 of FIGS. 2A, 2C, 2D) in addition to a pair of goggles that are specifically designed to protect the retinae of the eyes of the subject to be treated with the narrow-band infrared radiation from direct illumination at the wavelength(s) of the narrow-band infrared radiation. The protective shield generally includes a material that blocks the narrow-band infrared irradiation so that the narrow-band infrared irradiation can not spread to outside of the area where the narrow-band infrared radiation treatment is being performed. Any suitable material known in the art that blocks infrared irradiation can be employed for the protective shield of the invention. One suitable example of such materials is aluminum. The protective shield can be of either a hard material (e.g., metal plate) or a soft material (e.g., a cloth containing aluminum foil).

In yet another specific embodiment, as shown in FIGS. 2A and 2B, a kit of this invention comprises a pair of goggles 20 and radiation device 30. As shown in FIG. 2B, goggles 20 include one or more detection components 22. Radiation device 30 includes radiation source 31 and one or more detector(s) 34 that can detect or sense a signal from, or the presence of, the detection components of the protective goggles 22. In a further specific embodiment, detector(s) 34 is placed at radiation source 31 (e.g., radiation head) of radiation device 30. One example of this embodiment is shown in FIGS. 2A-2C. As shown in FIG. 2B, goggles 20 include one or more detection components 22. Detection component(s) 22 of goggles 20 can contain any component that can be detected or sensed by detector(s) 34. Alternatively, detector(s) 34 includes a component that can be activated by a signal transmitted from detection component(s) 22 of goggles 20.

In a further specific embodiment, detector(s) 34 can sense an area where the eyes of the subject would be placed, for example, an area facing the upper half of radiation source 31 (e.g., radiation head) of radiation device 30, and detect the presence or absence of goggles 20 by detecting the detection components 22 of goggles 20. In this embodiment, radiation source 31 of radiation device 30 can be activated by controller(s) 35 (which is in communication with detector(s) 34) only when detector(s) 34 detects or senses the presence of the detection components 22 of goggles 20 in the eye area of the subject's face.

In another, further specific embodiment, detector(s) 34 can scan the contour of the subject's face, detect the presence or absence of goggle 20 on the subject's face. In this embodiment, controller(s) 35 (which is in communication with detector(s) 34) activates radiation source 31 of radiation device 30 only when detector(s) 34 detects or senses the presence of goggles 20 on the subject's face.

In yet another specific embodiment, as shown in FIGS. 2A and 2B, a kit of this invention comprises protective shield 10, a pair of goggles 20 and radiation device 30 that is designed such that it cannot be activated without properly being connected to protective shield 10. In one specific example of such designs, protective shield 10 and radiation device 30 include one or more connecting spots 12 and 32, respectively. Radiation device 30 also includes one or more detectors 34. Detector(s) 34 detect or sense a signal from, or the presence of detection component(s) 22 of protective goggles 22. Features, including specific features, of protective shield 10, goggles 20 and detectors 34 of radiation device 30 are as described above.

In yet another specific embodiment, radiation device 30 further includes controller(s) 35 (see FIG. 2A) that controls the activation of the radiation source 31 of radiation device 30. The controller(s) 35 is in communication with detector(s) 34 and connecting spots of the radiation source and the protective shield 12 and 32, electronically, via signal(s) or via any other means known in the art. For example, controller(s) 35 can receive information (e.g., electronic information) as to the presence or absence of protective goggles from detector(s) 34.

In a further specific embodiment, controller(s) 35 receives information (e.g., electronic information), or signals, from detector(s) 34, determines whether or not connecting spots 12 and 32 are properly connected to each other, and controls the activation of the radiation source 31 of radiation device 30. For example, when detector(s) 34 detects or senses the presence of detection components 22 of protective goggles 20 in the proper location, that is, in the area facing the upper half of the radiation source 31 of the radiation device 30, or scans the subject's face and detects the presence of goggles 20 in the proper position, that is, the eye area of the subject's face, the controller(s) 35 receives this information from the detector(s) 34 and allows the activation of radiation source 31 of the radiation device 30. Alternatively, when the controller(s) 35 does not receive the information of the presence of goggles 20 in a proper position, the proper position as mentioned above, the controller(s) does not activate radiation source 31 of radiation device 30. For further example, when connecting spots 12 and 32 are connected properly to each other, controller(s) 35 allows the activation of the radiation source. Alternatively, when connecting spots 12 and 32 are not connected properly to each other, controller(s) 35 disallows the activation of radiation source 31.

Any suitable electronic sensor known in the art can be employed in the invention for the sensor(s) of detector(s) 34. Any suitable electronic controller known in the art can be employed in the invention for the controller(s) 35 of radiation device 30.

In yet another specific embodiment, a kit of this invention comprises protective shield 10 that includes one or connecting spots 12; a pair of goggles 20 that includes one or more detection component(s) 22; and radiation device 30 that includes radiation source 31, one or more connecting spots 32, one or more detector(s) 34, one or more controller(s) 35 (see FIG. 2A-2C). Features, including specific features of protective shield 10, goggles 20, radiation device 30, radiation source 31, connecting spots 12 and 32, detection components 22, detectors 34, and controller(s) 35 are as described above. FIG. 2D shows a schematic view of a subject under the narrow-band infrared radiation treatment with the kit including shield 10 that includes one or connecting spots 12; a pair of goggles 20 that includes one or more detection component(s) 22; and radiation device 30 that includes radiation source 31, one or more connecting spots 32, one or more detector(s) 34, and one or more controller(s) 35.

Although only few examples are illustrated in FIGS. 2A-2D, any other protective measure known in the art that can protect the eyes of the subject under the narrow-band infrared radiation treatment from any hazard from the narrow-band infrared radiation can also be employed in the invention.

A kit of the invention can further comprise a gel, cream, or lotion having at least 70% transparency at the narrow-band infrared radiation. Specific examples of suitable, transparent gels, creams or lotions are as described above for the methods of the invention. Specifically, the gel, cream or lotion included in the kit has at least 90% transparency at the narrow-band infrared radiation. More specifically, a transparent gel having at least 70% transparency, particularly at least 90% transparency, at the narrow-band infrared radiation is employed. Even more specifically, a water-based transparent gel having at least 70% transparency, particularly at least 90% transparency, at the narrow-band infrared radiation is employed.

The kits of the invention can be portable. Such a portable kit of the invention can be used as a home-therapy kit so that a user is the subject to be treated with the narrow-band infrared radiation. Alternatively, such a portable kit of the invention can also be used in a professional medical clinic for treating erythematotelangiectatic or papulopustular rosacea.

The invention is illustrated by the following examples which are not intended to be limiting in any way.

### EXEMPLIFICATION

### Example

### Treatment of Telangiectasia and Inflammatory Papules with Narrow Band Infrared Light at the Wavelength of 830 Nm

A light source for the phototherapy system consisted of a base and an irradiating head which emitted quasimonochromatic light of wavelength at 830 nm from adjustable planar arrays of LEDs. The irradiating head (Omnilux Plus™, Photo Therapeutics Ltd., Fazeley, UK) comprised five articulated panels containing 108 LEDs each, so that they could be adjusted to fit the contour of the patient's face optimally. The wavelength used was 830±5 nm with symmetrical peak. The irradiance was 55 mW/cm² at a distance of 1 to 10 centimeters from the light source. The radiant fluences, or doses, during a single treatment for twenty minutes were 66 J/cm².

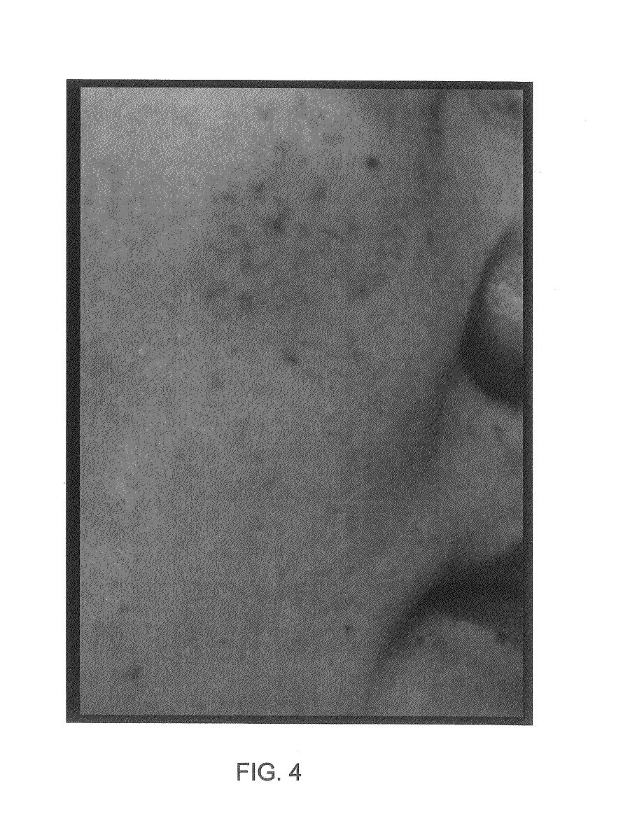

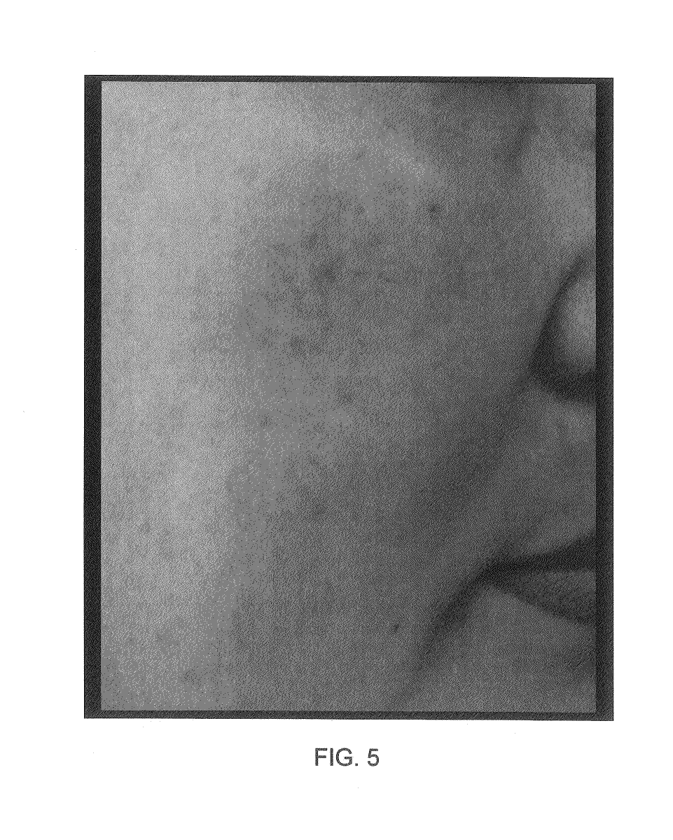

The patient was treated with this light source twice a week for four weeks at a three to four day interval between each session. The distance between the irradiating head and the patient's nose was about 3-5 cm. Goggles were worn to protect the retinae from direct illumination. Follow up evaluation was done at 3 months and 17 months after the final treatment. FIG. 3 shows a cheek of the patient prior to the narrow-band infrared radiation treatment. FIGS. 4 and 5 are follow-up photos of the cheek of the patient of FIG. 3 three months and seventeen months after the narrow-band infrared radiation treatment. As shown in FIGS. 4 and 5, telangiectasia on the patient's cheek was substantially reduced after the narrow-band infrared radiation treatment. Also, it was observed that inflammatory papules, found around the cheeks and the chin of the patient prior to the narrow-band infrared radiation treatment, were also reduced after the treatment (data not shown).

While this invention has been particularly shown and described with references to example embodiments thereof, it will be understood by those skilled in the art that various changes in form and details may be made therein without departing from the scope of the invention encompassed by the appended claims.

What is claimed is:

1. A method of treating erythematotelangiectatic rosacea or papulopustular rosacea in a subject, comprising exposing the subject's skin in need thereof to narrow-band infrared radiation at a wavelength(s) in a range of between 790 nm and 900 nm and having a band width of between 0 nm and 20 nm, in an effective dose to treat erythematotelangiectatic rosacea or papulopustular rosacea and essentially not to cause photothermolysis of the skin.

2. The method of claim 1, wherein the narrow-band infrared radiation has a power density between 1 mW/cm² and 100 mW/cm².

3. The method of claim 1, wherein the narrow-band infrared radiation has a power density between 1 mW/cm² and 75 mW/cm².

4. The method of claim 1, further comprising applying a layer of a gel, cream or lotion on the skin prior to the narrow-band infrared radiation, wherein the skin is exposed to the narrow-band infrared irradiation through the gel, cream or lotion layer.

5. The method of claim 4, wherein the gel, cream or lotion has at least 70% transparency at the narrow-band infrared radiation.

6. The method of claim 5, wherein the transparent gel is water-based.

7. The method of claim 6, wherein the water-based transparent gel comprises hyaluronic acid.

8. The method of claim 1, wherein the narrow-band infrared radiation is non-coherent narrow-band infrared radiation.

9. The method of claim 8, wherein the narrow-band infrared radiation is light-emitting diode radiation.

10. A method of treating erythematotelangiectatic rosacea or papulopustular rosacea in a subject, comprising exposing the subject's skin in need thereof to narrow-band infrared radiation at a wavelength(s) in a range of between 790 nm and 900 nm and having a band width of between 0.1 nm and 20 nm, in an effective dose to treat erythematotelangiectatic rosacea or papulopustular rosacea.

11. The method of claim 10, wherein the narrow-band infrared radiation has a power density between 1 mW/cm² and 100 mW/cm².

12. The method of claim 10, wherein the narrow-band infrared radiation has a power density between 1 mW/cm² and 75 mW/cm²

13. The method of claim 10, further comprising applying a layer of a gel, cream or lotion on the skin prior to the narrow-band infrared radiation, wherein the skin is exposed to the narrow-band infrared irradiation through the gel, cream or lotion layer.

14. The method of claim 13, wherein the gel, cream or lotion has at least 70% transparency at the narrow-band infrared radiation.

15. The method of claim 14, wherein the transparent gel is water-based.

16. The method of claim 15, wherein the water-based transparent gel comprises hyaluronic acid.

17. The method of claim 10, wherein the narrow-band infrared radiation is non-coherent narrow-band infrared radiation.

18. The method of claim 17, wherein the narrow-band infrared radiation is light-emitting diode radiation.

19. The method of claim 1, further comprising repeating exposure of the skin of the subject to the narrow-band infrared radiation at intervals of from 0.5 day to 10 days.

20. The method of claim 19, wherein the skin is exposed to the narrow-band infrared radiation one, two, three, four, five, six or seven times per week.

21. The method of claim 20, wherein the exposures are repeated for a duration of between one week and twelve weeks.

22. The method of claim 1, wherein the band width of the narrow-band infrared radiation is between 0.1 nm and 12 nm.

23. The method of claim 22, wherein the wavelength(s) is in a range between 800 nm and 860 nm.

24. The method of claim 23, wherein the narrow-band infrared radiation is at 830 nm.

25. The method of claim 1, wherein the skin is exposed to the narrow-band infrared radiation with the energy density of between 3 J/cm² and 180 J/cm² per single dose.

26. A kit, comprising:
a. a radiation device that comprises a radiation source generating narrow-band infrared radiation at a wavelength(s) in a range of between 790 nm and 900 nm, the narrow-band infrared radiation having a band width of between 0 nm and 20 nm and having a power density in a range of between 1 mW/cm² and 100 mW/cm²; and
b. a manual instructing a user how to use the narrow-band infrared radiation for narrow-band infrared irradiation treatment to treat erythematotelangiectatic rosacea or papulopustular rosacea of the skin of a subject.

27. The kit of claim 26, wherein the power density is between 1 mW/cm² and 75 mW/cm².

28. The kit of claim 27, wherein the power density is between 1 mW/cm² and 50 mW/cm².

29. The kit of claim 26, wherein the radiation source is a non-coherent radiation source.

30. The kit of claim 26, wherein the non-coherent radiation source is a light-emitting diode device.

31. The kit of claim 30, wherein the light-emitting diode device comprises a plurality of light-emitting diodes.

32. The kit of claim 26, wherein the manual comprises instructions about distance between the skin and the radiation source during the narrow-band infrared radiation treatment, duration time per single treatment of the narrow-band infrared radiation and frequency of the narrow-band infrared radiation treatment, and warning about conditions where the narrow-band infrared radiation treatment may affect the health of the skin.

33. The kit of claim 26, wherein the band width of the narrow-band infrared radiation is between 0.1 nm and 20 nm.

34. The kit of claim 33, wherein the band width of the narrow-band infrared radiation is between 0.1 nm and 12 nm.

35. The kit of claim 34, wherein the wavelength(s) is in a range between 800 nm and 860 nm.

36. The kit of claim 35, wherein the wavelength(s) is in a range between 825 nm and 835 nm.

37. The kit of claim 36, wherein the wavelength is 830 nm.

38. The kit of claim 26, further comprising a pair of goggles that have color and/or optical density to essentially block light at the wavelength(s) of the narrow-band infrared radiation.

39. The kit of claim 38, wherein the goggles comprise one or more detection components.

40. The kit of claim 26, wherein the radiation device comprises one or more detectors that detect the presence of the goggles.

41. The kit of claim 26, further comprising a protective shield, the protective shield comprising a material that blocks the narrow-band infrared radiation.

42. The kit of claim 41, wherein the protective shield further comprises one or more connecting spots that are configured to be connected to the radiation device.

43. The kit of claim 26, wherein the radiation device further comprises one or more connecting spots that are configured to be connected to the connecting spots of the protective shield.

44. The kit of claim 43, wherein the radiation device comprises a controller that is in communication with the detectors and controls the activation of the radiation source.

45. The kit of claim 44, wherein the controller of the radiation device determines whether or not the connecting spots of the protective shield and the connecting spots of the radiation device are connected to each other.

46. The kit of claim 26, further comprising a gel, cream or lotion having at least 70% transparency at the narrow-band infrared radiation.
